(19) United States
(12) Patent Application Publication
(10) Pub. No.: US 2013/0274661 A1
(43) Pub. Date: Oct. 17, 2013

(54) DRUG DELIVERY DEVICE WITH HOUSING COMPRISING FRANGIBLE ZONE

(71) Applicants: Axel Teucher, Frankfurt am Main (DE); Michael Jugl, Frankfurt am Main (DE)

(72) Inventors: Axel Teucher, Frankfurt am Main (DE); Michael Jugl, Frankfurt am Main (DE)

(73) Assignee: SANOFI-AVENTIS DEUTSCHLAND GMBH, Frankfurt am Main (DE)

(21) Appl. No.: 13/992,332

(22) PCT Filed: Dec. 20, 2011
(86) PCT No.: PCT/EP2011/073383

§ 371 Date: Jun. 7, 2013

(30) Foreign Application Priority Data
Dec. 21, 2010 (EP) 10196229.8

(51) Int. Cl. A61M 5/50

(52) U.S. Cl. CPC A61M 5/50
USPC 604/110

(57) ABSTRACT

The present invention relates to a drug delivery device for injecting a dose of a medicament, comprising of a cartridge holder adapted to house a cartridge filled with the medicament and comprising a displaceable piston, a body adapted to house a drive mechanism comprising a piston rod to be operably engaged with the piston of the cartridge for expelling a dose of the medicament, wherein the cartridge holder and the body are interconnected with each other and wherein the cartridge holder and/or the body comprise at least one separate fractionizing piece irreversibly detachable from the cartridge holder and/or from the body to irreversibly abrogate the interconnection of cartridge holder and body.

### CROSS REFERENCE TO RELATED APPLICATIONS

The present application is a U.S. National Phase Application pursuant to 35 U.S.C. §371 of International Application No. PCT/EP2011/073383 filed Dec. 20, 2011, which claims priority to European Patent Application No. 10196229.8 filed Dec. 21, 2010. The entire disclosure contents of these applications are herewith incorporated by reference into the present application.

### FIELD OF THE DISCLOSURE

The present invention relates to a drive mechanism for a drug delivery device that allows a user to select single or multiple doses of an injectable medicament and to dispense the set dosage of the medicament as well as to apply said medicament to a patient, preferably by injection. In particular, the present invention relates to such devices, which are handled by the patients themselves.

### BACKGROUND

Drug delivery devices allowing for multiple dosing of a required dosage of a liquid medicinal product, such as liquid medicaments, and further providing administering of the liquid to a patient, are as such well-known in the art.

Drug delivery devices of this kind have to meet a number of user specific requirements. For instance in case of those with diabetes, many users will be physically infirm and may also have impaired vision. Therefore, these devices need to be robust in construction, yet easy to use, both in terms of the manipulation of the parts and understanding by a user of its operation. Further, the dose setting must be easy and unambiguous and where the device is to be disposable rather than reusable, the device should be inexpensive to manufacture and easy to dispose. In order to meet these requirements, the number of parts and steps required to assemble the device and an overall number of material types the device is made from have to be kept to a minimum.

Typically, the medicament to be administered is provided in a cartridge having a displaceable piston or bung mechanically interacting with a piston rod of a drive mechanism of the drug delivery device. By way of the piston rod, thrust can be applied to the piston in distal direction and a certain amount of the medicinal fluid can be expelled from the cartridge.

Drug delivery devices, such like pen-type injectors further comprise multiple housing components, for instance a cartridge holder adapted to receive a cartridge filled with the medicament as well as a pen body housing or body adapted to receive and to house the drive mechanism which is to be operably engaged with the piston of the cartridge. In particular with disposable pen-type injectors, the entire drug delivery device is intended to be discarded after consumption or after use of the medicament stored in its cartridge.

Since the cartridge is typically made of glass or comparable material being inert to the medicament disposed therein, the cartridge and the housing and/or the functional components of the drug delivery device should be discarded or recycled in separate ways. Proper recycling or discarding of the drug delivery device therefore requires separation of the cartridge from the drug delivery device, which by virtue of its disposable design is not possible, because the drug delivery device is generally not intended to be disassembled.

It is therefore an object of the present invention to provide a drug delivery device of disposable type which provides an effective means to disassemble or to fractionize at least the housing components of the drug delivery device in order to enable separate recycling of the cartridge and the device components. It is a further object to provide a respective method for fractionizing or for decomposing the disposable drug delivery device in a well-defined and controlled way. Furthermore, it is intended to implement and/or to separate cartridge and device components in a cost-saving and efficient way, e.g. by only introducing minor amendments to the design of existing drug delivery devices.

### SUMMARY

The present invention provides a drug delivery device for injecting a dose of a medicament. The device comprises at least two housing components, for instance a cartridge holder and a body. The cartridge holder is adapted to house and to receive a cartridge filled with the medicament to be dispensed. The cartridge, typically designed as vial, carpule or ampoule comprises a barrel, typically made of glass or of comparable inert material which is sealed by way of a displaceable piston.

By exerting pressure to the piston, e.g. in distal direction, hence towards a patient, a respective pressure builds up inside the cartridge, thereby urging a well-defined dose of the liquid medicament through a dispensing outlet of the cartridge which is typically in fluid-communication with a piercing element, like an injection needle or cannula to be removably mounted on a distal end section of the cartridge holder. The body of the drug delivery device is typically adapted to house a drive mechanism comprising a piston rod or a drive ram to be operably engaged with the piston of the cartridge for exerting distally directed pressure to the cartridge for expelling a dose of the medicament.

The drug delivery device is preferably designed as a disposable device. Hence, cartridge holder and body are interconnected with each other in such a way, that a repeated disassembly and re-assembly is not possible. Therefore, if the medicament stored in the cartridge is used up or when the drug delivery device is only intended for non-regular but only temporary use, the entire device is intended to be discarded. Since most of the components of the housing and/or the drive mechanism comprise thermoplastic material or metal, a material separation, especially a separation of cartridge and device components should be provided in an easy and intuitive way.

For this purpose, the cartridge holder and/or the body comprise at least one fractionizing means that is adapted to irreversibly abrogate the interconnection of cartridge holder and body. By applying or activating the fractionizing means, cartridge holder and body are irreversibly disconnected from each other and may be separated accordingly. In this released and/or separated configuration, the cartridge disposed inside the cartridge holder can be removed from the cartridge holder and can be discarded or recycled in a separate way. Since the fractionizing means is adapted to irreversibly abrogate the interconnection of cartridge holder and body, misuse or operating errors, such as a user trying to replace an empty cartridge with a disposable drug delivery device, can be effectively prevented. The irreversible disassembly of the housing components, cartridge holder and body by means of the fractionizing means therefore enhances patient safety.

Furthermore the fractionizing means comprises at least one separate fractionizing piece that is irreversibly detachable from the cartridge holder and/or from the body. By detaching or removing the fractionizing piece from the cartridge holder or body, a mutual engagement, e.g. a positive engagement of cartridge holder and body can be abrogated and the interconnection of cartridge holder and body can be released. This way, cartridge holder and body can be separated from each other in order to disassemble the drug delivery device, e.g. for the purpose of waste or recycling separation. In particular, the cartridge can be taken out of the cartridge holder and can be discarded separately.

Mutual interconnection or engagement of fractionizing piece and cartridge holder or body, respectively, is designed such, that a re-assembly of fractionizing piece and cartridge holder or body is not possible. Once the fractionizing piece is detached or disengaged from the housing component of the drug delivery device, either from the cartridge holder or from the body, a re-attachment is effectively prevented. When the fractionizing piece is detached from the cartridge holder or from the body, the entire drug delivery device can only be discarded or recycled.

Replacement of an empty cartridge by a filled one as well as a further use of the device is effectively disabled and the device is rendered almost unusable, once the fractionizing means has been detached therefrom.

It is further conceivable that the fractionizing means and in particular the fractionizing piece is disintegrable itself. The fractionizing piece may be interconnectable with the body and/or with the cartridge holder in a positive-locking and/or force-fitting manner. The fractionizing piece may further comprise a predetermined breaking point or point of fracture in order to provide controlled and well-defined disintegration of the fractionizing piece itself. Once the fractionizing piece and/or its interconnection with either body and/or cartridge holder has been abrogated, e.g. by way of breaking and/or disintegrating the fractionizing piece itself, the mutual interconnection of body and cartridge holder can be irreversibly abrogated, thereby making the entire drug delivery device unusable.

Apart from a disintegration of the fractionizing piece it is also conceivable that the fractionizing piece is interconnected with or fastened to the body and/or to the cartridge holder by way of an adhesive which allows for controlled detachment of fractionizing piece from the body and/or from the cartridge holder. Since such detachment typically occurs after usage of the device, once the adhesive interconnection has broken, there will be no way to re-establish such interconnection and the device can only be discarded.

According to another preferred embodiment, the body and the cartridge holder are positively engaged by means of the fractionizing means. For instance, the fractionizing means may provide a snap-in or clipping feature by way of which the body and the cartridge holder remain interconnected as long as the fractionizing means remains inactive. As soon as the fractionizing means is activated, the positive engagement of body and cartridge holder is abrogated, such that body and cartridge holder can be separated from each other in a non-returning way.

According to another preferred embodiment, body and/or cartridge holder mutually overlap in an interface section when mutually interconnected. In said interface section, the body comprises a receptacle portion which is adapted to receive a corresponding insert portion of the cartridge holder. However, a diametrically opposite design is also conceivable, wherein a receptacle is provided at a proximal end of the cartridge holder while a distal end section of the pen body housing comprises an insert portion to be positioned therein.

In this way, cartridge holder and body can be arranged in an intertwined or interleaved manner providing a rather rigid and reliable mutual fastening of cartridge holder and body.

In a further preferred aspect, the fractionizing piece is fastened to an outside facing surface portion of the receptacle portion of the body. Furthermore, the fractionizing piece is engaged with a receptacle of the insert portion of the cartridge holder by intersecting the receptacle portion of the body with a radially inwardly extending protrusion. Hence, receptacle portion of the body as well as insert portion of the cartridge holder comprise mutually overlapping receptacles or through openings adapted to engage with radially inwardly extending protrusions of the fractionizing piece. Hence, by intersecting a through opening of the body's receptacle portion and by engaging with a receptacle of the insert portion arranged underneath, cartridge holder and body can be mutually and exclusively interconnected by way of the fractionizing piece. By removing the fractionizing piece, said interlock can be abrogated and released and as a consequence, body and cartridge holder can be separated from each other.

Alternatively, it is also conceivable, that body and cartridge are arranged in an intertwined or interleaved way, wherein the body comprises an insert piece at its distal end section adapted to be inserted into a corresponding receptacle portion arranged at a proximal end of the cartridge holder. With such an embodiment, the fractionizing piece is fastened to an outside facing surface portion of the receptacle portion of the cartridge holder and is further engaged with a corresponding receptacle disposed on an insert portion of the body. Irrespective on whether the cartridge holder is partially inserted into the body or whether the body is partially inserted into the cartridge holder, the fractionizing piece is arranged at the outside facing surface of a respective receptacle portion of the body-cartridge holder-interface.

In still another preferred embodiment, the fractionizing piece, at least when in its initial interlocking configuration, flushes with the outer circumference of the receptacle portion of body or cartridge holder. This way, unintentional displacement of the lug can almost be prevented since manipulation of the fractionizing piece requires a rather sophisticated lifting or gripping.

In still another aspect, the body and/or the cartridge holder are of substantially cylindrical geometry. Furthermore, the receptacle portion of body or cartridge holder comprises a circumferential receptacle adapted to receive an arc-shaped fractionizing piece. The receptacle may comprise a rim-like structure and preferably corresponds to the shape and geometry of the at least one fractionizing piece.

Typically, the outside facing receptacle at least partially surrounds the body or the cartridge holder. Preferably, the fractionizing means comprises at least two arc-shaped fractionizing pieces to be arranged on diametrically opposite sides of the receptacle portion of the body-cartridge holder-interface. Since the fractionizing pieces substantially flush with the adjacent surface section of the receptacle portion, unintentional lifting or removing of the fractionizing pieces is effectively prevented. Hence, by adapting the shape of the fractionizing piece to the outer shape of the body, the fractionizing pieces do not provide any kind of gripping or disengaging structure.

By making use of the fractionizing means, the drug delivery device can be disassembled and fractionized, such that at least the cartridge, typically comprising a glass barrel can be removed from the cartridge holder and can be discarded or recycled separately.

It is even of advantage, when the fractionizing piece is kept in an engagement configuration with the body and/or with the cartridge holder by means of an adhesive cover or foil. This way, detaching of the fractionizing piece from the receptacle portion of the body-cartridge holder-interface requires first a removal of the adhesive cover or foil.

It may be of further benefit, when the fractionizing piece is permanently attached to the adhesive cover or foil. This way, the adhesive cover or foil inherently provides a detaching means to detach the fractionizing piece from the receptacle portion of either cartridge holder or body. By simply removing the adhesive cover or foil, the fractionizing piece or several fractionizing pieces will be automatically detached, such that after removal of the adhesive cover or foil cartridge holder and body can be separated from each other.

In still another aspect, the drug delivery device is readily equipped with a cartridge positioned and fixed by the cartridge holder, wherein the cartridge is filled with the medicament to be dispensed. Moreover, the drive mechanism is already operably engaged with the piston of the cartridge when the drug delivery device is delivered to the end-customer.

Finally, the various components of the drug delivery device, in particular its cartridge and its housing or the functional components of its drive mechanism are intended to be separately discarded after consumption or use of the medicament.

In still another and independent aspect, the invention further relates to a method of fractionizing a drug delivery device after its use, wherein the drug delivery device comprises at least a body and a cartridge holder that are interconnected in an interface section in a mutually interleaved manner. In said interface section, a receptacle portion of body or cartridge holder receives an insert portion of the cartridge holder or body, respectively. In particular the method is applicable to a drug delivery device as described above.

The method of fractionizing the drug delivery device comprises the steps of irreversibly removing at least one fractionizing piece from a housing component of the drug delivery device. The fractionizing piece is preferably removed from the outer circumference of the receptacle portion of cartridge holder or body. In the next step, body and cartridge holder are separated from each other in order to provide access to the cartridge disposed in the cartridge holder. Thereafter, the cartridge is removed from the cartridge holder and is discarded or recycled separately from the housing or functional components of the drug delivery device.

This way, even a disposable drug delivery device, such like a pen-type injector intended to be discarded after usage can become subject to an environmentally friendly discarding- or recycling process.

The term “medicament”, as used herein, means a pharmaceutical formulation containing at least one pharmaceutically active compound,

wherein in one embodiment the pharmaceutically active compound has a molecular weight up to 1500 Da and/or is a peptide, a protein, a polysaccharide, a vaccine, a DNA, a RNA, a antibody, an enzyme, an antibody, a hormone or an oligonucleotide, or a mixture of the above-mentioned pharmaceutically active compound,

wherein in a further embodiment the pharmaceutically active compound is useful for the treatment and/or prophylaxis of diabetes mellitus or complications associated with diabetes mellitus such as diabetic retinopathy, thromboembolism disorders such as deep vein or pulmonary thromboembolism, acute coronary syndrome (ACS), angina, myocardial infarction, cancer, macular degeneration, inflammation, hay fever, atherosclerosis and/or rheumatoid arthritis,

wherein in a further embodiment the pharmaceutically active compound comprises at least one peptide for the treatment and/or prophylaxis of diabetes mellitus or complications associated with diabetes mellitus such as diabetic retinopathy,

wherein in a further embodiment the pharmaceutically active compound comprises at least one human insulin or a human insulin analogue or derivative, glucagon-like peptide (GLP-1) or an analogue or derivative thereof, or exedin-3 or exedin-4 or an analogue or derivative of exedin-3 or exedin-4.

Insulin analogues are for example Gly(A21), Arg(B31), Arg(B32) human insulin; Lys(B3), Glu(B29) human insulin; Lys(B28), Pro(B29) human insulin; Asp(B28) human insulin; human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Insulin derivates are for example B29-N-myristoyl-des(B30) human insulin; B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N—(N-palmitoyl-Y-glutamyl)-des(B30) human insulin; B29-N—(N-lithocholyl-Y-glutamyl)-des(B30) human insulin; B29-N-(ω-carboxyheptadecanoyl)-des(B30) human insulin and B29-N-(ω-carboxyheptadecanoyl) human insulin.

Exendin-4 for example means Exendin-4(1-39), a peptide of the sequence H-His-Gly-Glu-Gly-Thr-Phe-Thr-Ser-Asp-Leu-Ser-Lys-Gln-Met-Glu-Glu-Glu-Ala-Val-Arg-Leu-Phe-Ile-Glu-Trp-Leu-Lys-Asn-Gly-Gly-Pro-Ser-Ser-Gly-Ala-Pro-Pro-Pro-Ser-NH2.

Exendin-4 derivatives are for example selected from the following list of compounds:

### H-(Lys)4-des Pro36, des Pro37 Exendin-4(1-39)-NH2,

### H-(Lys)5-des Pro36, des Pro37 Exendin-4(1-39)-NH2,

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39); or

### des Pro36 [Asp28] Exendin-4(1-39),

### des Pro36 [IsoAsp28] Exendin-4(1-39),

### des Pro36 [Met(O)14, Asp28] Exendin-4(1-39),

### des Pro36 [Met(O)14, IsoAsp28] Exendin-4(1-39),

### des Pro36 [Trp(O2)25, Asp28] Exendin-4(1-39),

### des Pro36 [Trp(O2)25, IsoAsp28] Exendin-4(1-39),

### des Pro36 [Met(O)14 Trp(O2)25, Asp28] Exendin-4(1-39),

### des Pro36 [Met(O)14 Trp(O2)25, IsoAsp28] Exendin-4(1-39),

wherein the group -Lys6-NH2 may be bound to the C-terminus of the Exendin-4 derivative;
or an Exendin-4 derivative of the sequence

### H-(Lys)6-des Pro36 [Asp28] Exendin-4(1-39)-Lys6-NH2,

### des Asp28 Pro36, Pro37, Pro38Exendin-4(1-39)-NH2,

### H-(Lys)6-des Pro36, Pro38 [Asp28] Exendin-4(1-39)-NH2,

### H-Asn-(Glu)5des Pro36, Pro37, Pro38 [Asp28] Exendin-4(1-39)-NH2,

### des Pro36, Pro37, Pro38 [Asp28] Exendin-4(1-39)-(Lys)6-NH2,

### H-(Lys)6-des Pro36, Pro37, Pro38 [Asp28] Exendin-4(1-39)-(Lys)6-NH2,

### H-Asn-(Glu)5-des Pro36, Pro37, Pro38 [Asp28] Exendin-4(1-39)-(Lys)6-NH2,

### H-(Lys)6-des Pro36 [Trp(O2)25, Asp28] Exendin-4(1-39)-Lys6-NH2,

### H-des Asp28 Pro36, Pro37, Pro38 [Trp(O2)25] Exendin-4(1-39)-NH2,

### H-(Lys)6-des Pro36, Pro37, Pro38 [Trp(O2)25, Asp28] Exendin-4(1-39)-NH2,

### H-Asn-(Glu)5-des Pro36, Pro37, Pro38 [Trp(O2)25, Asp28] Exendin-4(1-39)-NH2,

### des Pro36, Pro37, Pro38 [Trp(O2)25, Asp28] Exendin-4(1-39)-(Lys)6-NH2,

### H-(Lys)6-des Pro36, Pro37, Pro38 [Trp(O2)25, Asp28] Exendin-4(1-39)-(Lys)6-NH2,

### H-Asn-(Glu)5-des Pro36, Pro37, Pro38 [Trp(O2)25, Asp28] Exendin-4(1-39)-(Lys)6-NH2,

### H-(Lys)6-des Pro36 [Met(O)14, Asp28] Exendin-4(1-39)-Lys6-NH2,

### des Met(O)14 Asp28 Pro36, Pro37, Pro38 Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Asp28] Exendin-4(1-39)-NH2,

### des Pro36, Pro37, Pro38 [Met(O)14, Asp28] Exendin-4(1-39)-(Lys)6-NH2,

### H-(Lys)6-des Pro36, Pro37, Pro38 [Met(O)14, Asp28] Exendin-4(1-39)-(Lys)6-NH2,

### H-Asn-(Glu)5 des Pro36, Pro37, Pro38 [Met(O)14, Asp28] Exendin-4(1-39)-(Lys)6-NH2,

### H-Lys6-des Pro36 [Met(O)14, Trp(O2)25, Asp28] Exendin-4(1-39)-Lys6-NH2,

### H-des Asp28 Pro36, Pro37, Pro38 [Met(O)14, Trp(O2)25] Exendin-4(1-39)-NH2,

### H-(Lys)6-des Pro36, Pro37, Pro38 [Met(O)14, Asp28] Exendin-4(1-39)-NH2,

### H-Asn-(Glu)5-des Pro36, Pro37, Pro38 [Met(O)14, Trp(O2)25, Asp28] Exendin-4(1-39)-NH2,

### des Pro36, Pro37, Pro38 [Met(O)14, Trp(O2)25, Asp28] Exendin-4(1-39)-(Lys)6-NH2,

### H-(Lys)6-des Pro36, Pro37, Pro38 [Met(O)14, Trp(O2)25, Asp28] Exendin-4(S1-39)-(Lys)6-NH2,

### H-Asn-(Glu)5-des Pro36, Pro37, Pro38 [Met(O)14, Trp(O2)25, Asp28] Exendin-4(1-39)-(Lys)6-NH2;

or a pharmaceutically acceptable salt or solvate of any one of the afore-mentioned Exedin-4 derivative.

Hormones are for example hypophysis hormones or hypothalamus hormones or regulatory active peptides and their antagonists as listed in Rote Liste, ed. 2008, Chapter 50, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, Goserelin.

A polysaccharide is for example a glucosaminoglycane, a hyaluronic acid, a heparin, a low molecular weight heparin or an ultra low molecular weight heparin or a derivative thereof, or a sulphated, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium.

Pharmaceutically acceptable salts are for example acid addition salts and basic salts. Acid addition salts are e.g. HCl or HBr salts. Basic salts are e.g. salts having a cation selected from alkali or alkaline, e.g. Na+, or K+, or Ca2+, or an ammonium ion N+(R1)(R2)(R3)(R4), wherein R1 to R4 independently of each other mean: hydrogen, an optionally substituted C1-C6-alkyl group, an optionally substituted C2-C6-alkenyl group, an optionally substituted C6-C10-aryl group, or an optionally substituted C6-C10-heteroaryl group. Further examples of pharmaceutically acceptable salts are described in “Remington's Pharmaceutical Sciences” 17. ed. Alfonso R. Gennaro (Ed.), Mark Publishing Company, Easton, Pa., U.S.A., 1985 and in Encyclopedia of Pharmaceutical Technology.

Pharmaceutically acceptable solvates are for example hydrates.

It will be further apparent to those skilled in the pertinent art that various modifications and variations can be made to the present invention without departing from the spirit and scope of the invention. Further, it is to be noted, that any reference signs used in the appended claims are not to be construed as limiting the scope of the present invention.

### BRIEF DESCRIPTION OF THE DRAWINGS

In the following, preferred embodiments of the invention will be described in greater detail by making reference to the Figures in which:

FIG. 1 schematically illustrates a pen-type injector,

FIG. 2 shows the injector according to FIG. 1 with several of its components in an exploded view,

FIG. 3 shows the cartridge holder in an enlarged view and

FIG. 4 illustrates a distal portion of a proximal body housing component,

FIG. 5a perspectively illustrates a fractionizing piece,

FIG. 5b shows the fractionizing piece according to FIG. 5a in another perspective view and

FIG. 6 shows two separate fractionizing pieces adhered to an adhesive cover or foil.

### DETAILED DESCRIPTION

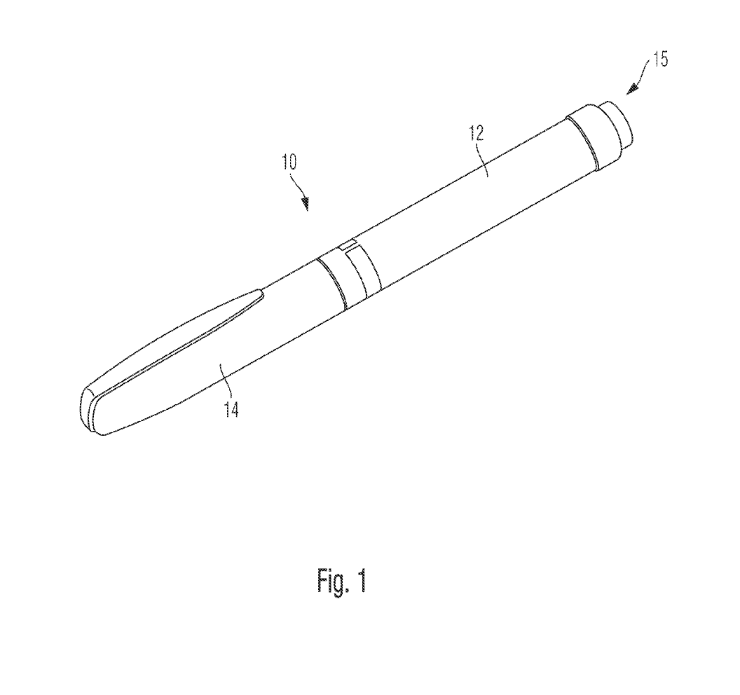

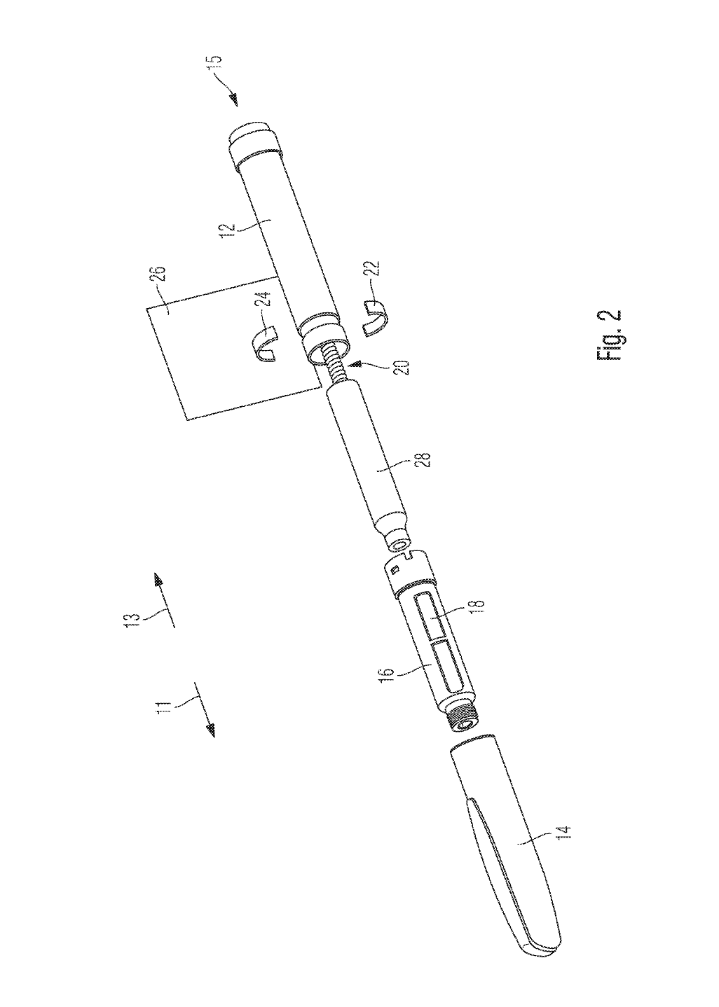

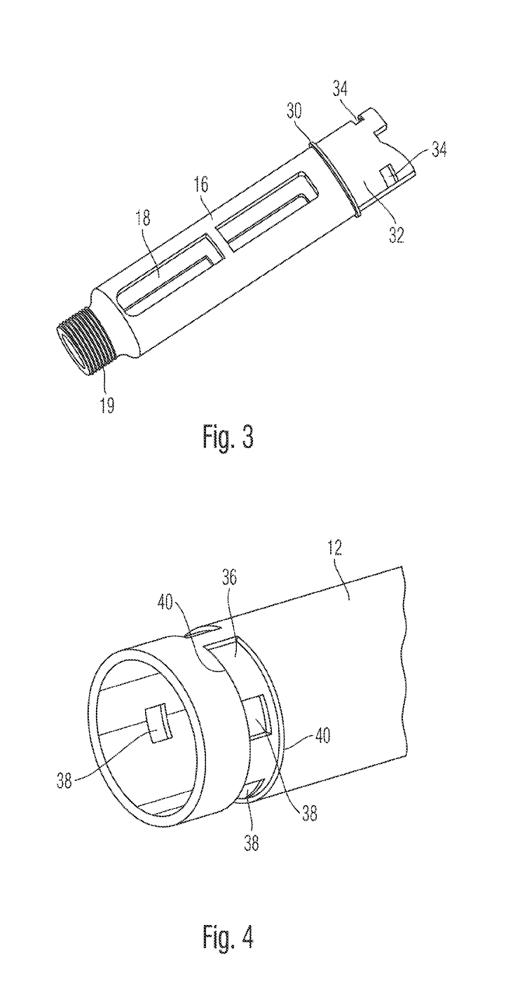

The drug delivery device 10 as depicted in FIGS. 1 and 2 comprises a pen body housing 12 connected with a cartridge holder section 16, in which a cartridge 28 is disposed. When assembled, the cartridge 28 remains visible through an inspection window 18 of the cartridge holder 16 in order to inspect its filling level. As shown in FIG. 3, the cartridge holder 16 comprises a threaded socket 19 at its distal end section adapted to receive a correspondingly threaded needle assembly having an injection needle intended to pierce a distally located sealing member of the cartridge 28, which is typically designed as a septum.

Opposite its distal outlet, the cartridge 28 comprises a displaceable piston to operably engage with the piston rod or drive ram 20 of a drive mechanism that is housed in the body 12. At a proximal end of the body 12, a dose button 15 is located allowing to manipulate and to control dose setting and dose dispensing of the drug delivery device 10. Body 12 and cartridge holder 16 are interconnected by forming an interface in an interleaved and mutually overlapping manner. In the illustrated embodiment, the distal end of the body 12 comprises a receptacle adapted to receive a proximally located insert portion 32 of the cartridge holder 16.

The illustrated drug delivery device 10 is preferably of disposable type. Hence, when the device 10 is not intended for regular but only temporary use, the entire device 10 should be discarded when a treatment with the medicament has terminated. Otherwise, for patients regularly using such pen-type injectors 10, the entire device 10 is to be discarded when the medicament provided in the cartridge 28 is used up.

In FIG. 2 the distal direction is denoted with reference numeral 11 whereas the opposite, proximal direction is characterized by the arrow 13.

The drive mechanism featuring an axially displaceable piston rod 20 is to be operably engaged with a proximally located piston to be displaced in distal direction with respect to the barrel of the cartridge 28, which is typically made of glass or some comparable material inert to the medicament disposed therein.

Adjacent to its insert portion 32, the cartridge holder 16 comprises an annular rim 30 at a proximal portion that serves to but against a distally located end face of the body 12 as depicted in FIG. 4.

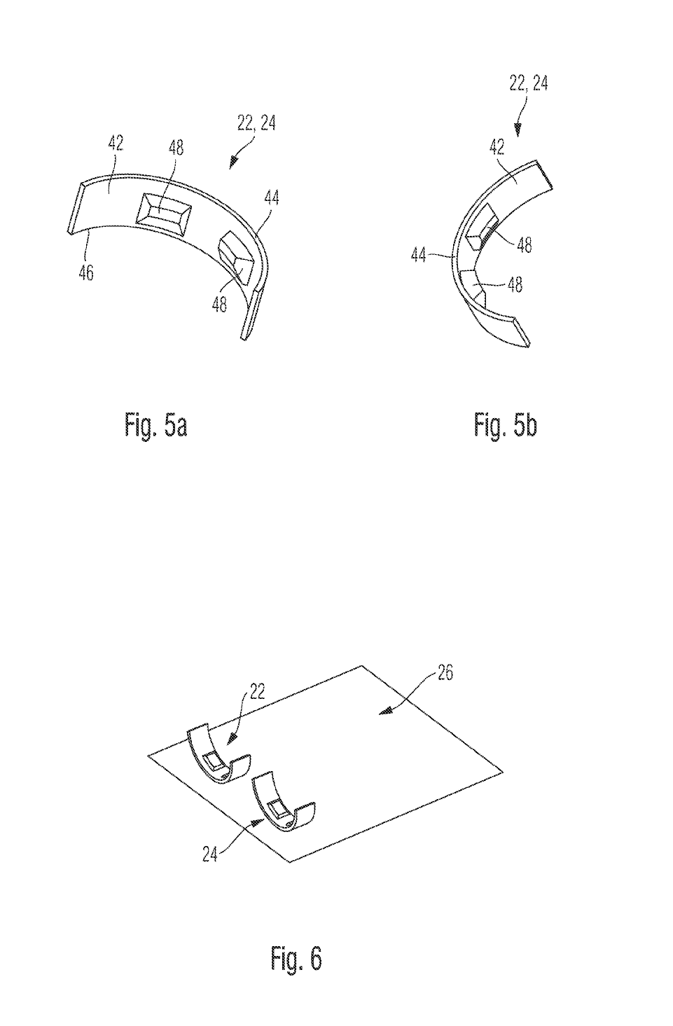

As further illustrated there, the body 12 which is also of cylindrical geometry comprises a receptacle portion at its distal end section which is adapted to receive the proximally located insert portion 32 of the cartridge holder 16. Body 12 and insert portion 32 of the cartridge holder 16 further comprise mutually corresponding through openings or receptacles 34, 38 at least partially overlapping when cartridge holder 16 and body 12 are interconnected as indicated in the sketch of FIG. 1. The distally located receptacle portion of the body 12 further comprises a circumferential receptacle 36 adapted to receive an arc-shaped fractionizing piece 22, 24, separately illustrated in FIGS. 5a and 5b.

The fractionizing pieces 22, 24 comprises two radially inwardly protruding prongs 48 adapted to intersect the through opening 38 disposed in the groove-like receptacle 36 of the distally located receiving portion of the body 12. The radial extension of said prongs 48 is preferably larger than the margins of the through opening 38, such that radially inwardly pointing end sections of the prongs 48 engage with the through opening or receptacle 34 of the insert piece 32 of the cartridge holder 16.

This way, the fractionizing pieces 22, 24 provide a positive interlock for the cartridge holder 16 and the body 12. The fractionizing pieces 22, 24 comprise an arc-shaped rim 42 having a geometry that matches and corresponds with the groove-like receptacle 36 provided at the distal section of the body 12. Moreover, the rim 42 comprises oppositely located edge portions 44, 46, by way of which an axial abutment of the fractionizing pieces 22, 24 with respect to the body 12 can be attained.

In assembly or interlock configuration, the distal and proximal edges 44, 46 of the fractionizing piece 22, 24 abut with correspondingly shaped distal and proximal edge portions 40 of the circumferential receptacle 36 provided at a distal portion of the body 12. Here, the mutually corresponding edges 44, 46 of the fractionizing piece 22, 24 and the corresponding edge 40 of the body's receptacle 36 are adapted to transfer axial forces or axial stress that may arise during a dose dispensing action.

This way, axial stress applied via the piston rod 20 to the cartridge 28 and hence to the cartridge holder 16 may transfer via the receptacle 34 to the prongs 48 of the fractionizing pieces 22, 24, which in turn transfer said mechanical stress via the contact surfaces 44, 46 to the body 12.

In FIG. 6 an unwrapped adhesive cover or foil 26 is illustrated which is intended to keep the two diametrically oppositely located fractionizing pieces 22, 24 in engagement position on the pen body housing 12. By way of the adhesive cover or foil 26, access to the fractionizing pieces 22, 24 is effectively inhibited.

Disassembly of the drug delivery device 10 therefore initially requires detachment of the adhesive cover or foil 26, which may disintegrate when pulled off from the body 12. After removal of the protective and adhesive cover or foil 26, the fractionizing pieces 22, 24 may be removed from the body 12. However, it is also conceivable, that the fractionizing pieces 22, 24 permanently adhere to the adhesive cover or foil 26. As a consequence, the fractionizing pieces 22, 24 may automatically separate from the body 12 in the course of a detachment of the adhesive foil or cover 26 from the body 12.

The adhesive component to attach the foil 26 to the body 12 as well as the composition of the foil 26 itself may be chosen such that the foil 26 is disabled to be re-attached to the body 12 once it has been removed therefrom. This way, a re-assembly of the drug delivery device 10 by re-inserting the cartridge holder 16 into the distal receptacle portion of the body 12 and by re-attaching the interlocking fractionizing pieces 22, 24 is effectively inhibited.

After disassembly of cartridge holder 16 and body 12, the device 10 can only be discarded as intended by the manufacturer. Unintentional reuse of the device which might endanger the patient's health can be therefore effectively prevented.

1-13. (canceled)

14. Drug delivery device for injecting a dose of a medicament, comprising:
a cartridge holder housing a cartridge filled with the medicament and comprising a displaceable piston,
a body housing a drive mechanism comprising a piston rod to be operably engaged with the piston of the cartridge for expelling a dose of the medicament,
wherein the body and/or the cartridge holder mutually overlap in an interface section, in which the body comprises a receptacle portion which is adapted to receive an insert portion of the cartridge holder, or vice versa, and wherein the cartridge holder and the body are interconnected with each other and
wherein the cartridge holder and/or the body comprise at least one fractionizing means by way of which the body and the cartridge holder are positively engaged, and wherein the fractionizing means is adapted to irreversibly abrogate the interconnection of cartridge holder and body to remove the cartridge from the cartridge holder, and
wherein the fractionizing means further comprises at least one separate fractionizing piece irreversibly detachable from the cartridge holder and/or from the body,
wherein the receptacle portion of body or cartridge holder is at least partially provided with an adhesive cover or foil covering the fractionizing piece, and
wherein the fractionizing piece is kept in an engagement configuration with the body and/or with the cartridge holder by means of an adhesive cover or foil.

15. The drug delivery device according to claim 14, wherein the fractionizing piece is fastened to an outside facing surface portion of the receptacle portion of the body and is further engaged with a receptacle of the insert portion of the cartridge holder by intersecting the receptacle portion of the body with a radially inwardly extending protrusion.

16. The drug delivery device according to claim 14, wherein the fractionizing piece flushes with the outer circumference of the receptacle portion of the body when in interlock configuration.

17. The drug delivery device according to claim 14, wherein the body and/or the cartridge holder are of substantially cylindrical geometry and wherein the receptacle portion of body or cartridge holder comprises a circumferential receptacle adapted to receive an arc shaped fractionizing piece.

18. The drug delivery device according to claim 17, wherein the circumferential receptacle comprises at least one through opening to receive a radially inwardly directed protrusion of the fractionizing piece.

19. The drug delivery device according to claim 14, wherein the fractionizing piece is permanently attached to the adhesive cover or foil.

20. A method of fractionizing a drug delivery device according to claim 14 after its use, comprising the steps of:
irreversibly removing the fractionizing piece from a receptacle portion of the body or cartridge holder which receives an insert portion of the cartridge holder or body by removing the adhesive cover or foil from the cartridge holder or body thereby bringing cartridge holder and body into a release configuration,
separating body and cartridge holder in order to gain access to the cartridge disposed therein,
removing the cartridge from the cartridge holder and discarding the cartridge separate from the housing components of the drug delivery device.
